(19) United States
(12) Patent Application Publication
(10) Pub. No.: US 2025/0172560 A1
(43) Pub. Date: May 29, 2025

(54) METHODS OF CLASSIFYING RESPONSE TO IMMUNOTHERAPY FOR CANCER

(71) Applicant: Arizona Board of Regents on behalf of Arizona State University, Scottsdale, AZ (US)

(72) Inventor: Stephen Johnston, Tempe, AZ (US)

(21) Appl. No.: 18/753,713

(22) Filed: Jun. 25, 2024

Related U.S. Application Data
(63) Continuation of application No. 16/647,434, filed on Mar. 13, 2020, now Pat. No. 12,025,615.
(60) Provisional application No. 62/559,337, filed on Sep. 15, 2017.

(51) Int. Cl. G01N 33/574; C07K 14/705; C07K 16/28; G01N 33/543; G16B 40/00; G16H 10/40; G16H 10/60; G16H 20/00; G16H 50/00; G16H 50/20; G16H 50/30; G16H 50/70

(52) U.S. Cl. CPC G01N 33/574; C07K 16/2818; C07K 16/2827; G01N 33/543; G16B 40/00; G16H 10/40; G16H 20/00; G16H 50/00; G16H 50/20; G16H 50/30; G16H 50/70; C07K 14/70503; C07K 14/70521; C07K 16/2803; C07K 2317/76; G01N 2800/52; G01N 2800/60; G16H 10/60

(57) ABSTRACT

Provided herein are methods for classifying how a subject having a cancer will respond to immunotherapeutic (IT) therapy based on the subject's immunosignature or frameshift signature. Also provided herein are methods for classifying a subject having a cancer as having a good prognosis or a poor prognosis based on the subject's immunosignature or frameshift signature.

### CROSS-REFERENCE TO RELATED APPLICATIONS

This application is a continuation of U.S. application Ser. No. 16/647,434, filed Mar. 13, 2020, which is a U.S. national phase application of PCT international application No. PCT/US2018/050827 filed Sep. 13, 2018, which claims the benefit of U.S. Provisional Patent Application No. 62/559,337, filed Sep. 15, 2017; this application claims the benefit of each of the foregoing and each is incorporated herein by reference as if set forth in its entirety.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### STATEMENT REGARDING FEDERALLY SPONSORED R&D

Not applicable.

### BACKGROUND

### Field

This application relates to methods and compositions that allow the prediction of a response of a patient to therapeutic treatment with immunomodulators such as checkpoint inhibitors. The prediction could be relative to the therapeutic response or side-effects. The application also bears on prognosis in general for cancer outcomes.

### Description of the Related Art

The treatment of cancer has been transformed over the last few years by the development of immunotherapeutics (IT). Immunotherapeutics include checkpoint inhibitors which enhance the natural anti-tumor immune response or help augment personal, neoepitope vaccines. Immunotherapeutics also include cell-based therapies such as adoptive T-cell therapies in which large numbers of a patient's T-cells are modified ex vivo to express chimeric antigen receptors (CARs) and returned to the patient as engineered T-cells.

However, this revolution has limitations. Immunotherapeutic treatments are very expensive, in some cases between $150,000 and $450,000 or more. On average only about 25% of patients who receive immunotherapy have a positive clinical response to the treatment. In addition, there can be severe side-effects, including death.

This has spurred an effort to identify biomarkers that could distinguish patients more likely to have a positive response and to predict which patients would have deleterious side effects. Initially the level of expression of PDL-1 on the tumor cells was used as a correlate of protection. This is currently being used as a biomarker in spite of its low predictive value. As the DNA sequences of more patient tumors were analyzed, it was realized that a more relevant correlate was the level of neoantigen mutations in the DNA of the tumor. This makes sense in that the neoantigens drive the anti-tumor immune response. In special cases of microsatellite instability (MSI) the mutations in the tumor that create frameshift neoantigens are strong predictors of a positive response to therapy. The correlation was so strong that for the first time the FDA awarded Merck (now also BMS) a blanket approval to treat any cancer with MSI with anti-PD-1 antibody. However, MSI is only prevalent in a small percentages of cancer. For most cancers, the hope is that Total Mutation Burden (TMB) will be the biomarker of choice for deciding who might respond to therapy. In this protocol a portion of the DNA from a tumor biopsy is sequenced and the total number of mutations tabulated. This number is used to predict response. Though it has had marginal predictive value, it currently is the best alternative. Because of the cost and that only some tumors yield enough good DNA for sequencing, there is interest in sequencing the tumor free DNA in the blood. This carries with it other problems, notably the blood dilution issue.

A basic problem with TMB and related DNA-based approaches is that what is recorded (mutations) is far removed from the relevant biological activity—namely the immune response elicited by a neoantigen. A checkpoint inhibitor is presumed to work by blocking the down-regulatory signals on T-cells, allowing the immune response that the tumor has already elicited to be more effective. TMB measures any mutation which is indirectly related to neoantigen creating mutations. Only about 1% non-synonymous mutations are potentially immunogenic. To be immunogenic on the tumor they have to be expressed at the RNA level, be processed by the proteasome and end up on an MHC molecule—all of which steps have a finite probability of success. Given this complexity of relationships it does not seem surprising that TNIB is only weakly predictive of a positive response to checkpoint treatment. Accordingly, there is a pressing need for biomarkers that are more closely related to the biologically relevant response for screening to predict response to immunotherapeutics.

### SUMMARY

This disclosure relates to the application of methods and compositions for classification and characterization of subjects with respect to their likely response to treatment with an immunotherapeutic. In particular, this disclosure relates to the application of two peptide array formats—immunosignature (IMS) and frameshift signature (FS)—to the classification and characterization of subjects with respect to their likely response to treatment with a checkpoint inhibitors or other immunotherapeutics (IT).

In a first aspect, provided in this disclosure is a method of classifying how a subject having cancer may respond to treatment with an immunotherapeutic, the method comprising: (a) contacting a biological sample from the subject to a frameshift peptide array comprising a plurality of tumor-associated frameshift peptides; (b) detecting the presence or absence of antibodies having affinity to one or more of the frameshift peptides in the contacted biological sample; (c) quantifying a level of the antibodies having affinity to one or more of the frameshift peptides to form a frameshift signature of the subject; and (d) comparing the frameshift signature of the subject to one or more frameshift signature standards comprising a frameshift signature of one or more subjects known to respond to an immunotherapeutic treatment, and wherein the subject is classified as being likely to respond to treatment with the immunotherapeutic based on the comparison. The immunotherapeutic can be selected from the group consisting of a CTLA-4 inhibitor, a PD-L1 inhibitor, and a PD-1 inhibitor. The comparison can be predictive of the clinical outcome of the immunotherapeutic treatment in the subject. The biological sample can be a blood or tissue sample. The blood sample can be a peripheral blood sample. The cancer can be selected from the group consisting of breast cancer, renal cell carcinoma, lung cancer, and melanoma. The subject can be a mammal. The mammal can be a human. The mammal can be a canine. In some cases, the method further comprises creating a record indicating the subject is likely to respond to the immunotherapeutic treatment based on the frameshift signature. The record can be created on a computer readable medium.

In a further aspect, provided herein is a method for classifying a subject having a cancer as having a good prognosis or a poor prognosis, the method comprising the steps of: (a) contacting a biological sample from the subject to a frameshift array comprising a plurality of tumor-associated frameshift peptides; (b) detecting the presence or absence of antibodies having affinity to one or more of the frameshift peptides in the contacted biological sample; (c) quantifying a level of the antibodies having affinity to one or more of the frameshift peptides to form a frameshift signature of the subject; and (d) classifying the subject as having a good prognosis or a poor prognosis based on comparison of the subject's frameshift signature to one or more frameshift signature standards comprising a frameshift signature of one or more subjects known to respond to an immunotherapeutic treatment, wherein the good prognosis indicates that said subject is expected to have a favorable response to an immunotherapeutic treatment, and wherein the poor prognosis indicates that said subject is expected to have an unfavorable response to an immunotherapeutic treatment. The immunotherapeutic can be selected from the group consisting of a CTLA-4 inhibitor, a PD-L1 inhibitor, and a PD-1 inhibitor. The comparison can be predictive of the clinical outcome of the immunotherapeutic treatment in the subject. The biological sample can be a blood or tissue sample. The blood sample can be a peripheral blood sample. The cancer can be selected from the group consisting of breast cancer, renal cell carcinoma, lung cancer, and melanoma. The subject can be a mammal. The mammal can be a human. The mammal can be a canine. In some cases, the method further comprises creating a record indicating the subject is likely to respond to the immunotherapeutic treatment based on the frameshift signature. The record can be created on a computer readable medium.

In a further aspect, provided herein is a method of classifying whether a subject might experience an immune-related adverse event in response to treatment, the method comprising the steps of: (a) contacting a biological sample from the subject to a frameshift array comprising a plurality of tumor-associated frameshift peptides; (b) detecting the presence or absence of antibodies having affinity to one or more of the frameshift peptides in the contacted biological sample; (c) quantifying a level of the antibodies having affinity to one or more of the frameshift peptides to form a frame shift signature of the subject; and (d) classifying the subject as having a high likelihood of experiencing an adverse event based on comparison of the subject's frameshift binding pattern to one or more standards, wherein the signature distinguishes a person likely to have an adverse event from those that are unlikely to have an adverse event in response to immunotherapy. The immunotherapeutic can be selected from the group consisting of a CTLA-4 inhibitor, a PD-L1 inhibitor, and a PD-1 inhibitor. The biological sample can be a blood or tissue sample. The blood sample can be a peripheral blood sample. The subject can be a mammal. The mammal can be a human. The mammal can be a canine. The method can further comprise creating a record indicating the subject is likely to experience an adverse event in response to the immunotherapeutic treatment based on the frameshift signature. The record can be created on a computer readable medium.

In another aspect, provided in this disclosure is a method of classifying a subject having cancer as likely to respond to an IT treatment. As described herein, the method can comprise or consist essentially of: (a) contacting a biological sample from the subject to a plurality of peptides capable of off-target binding to at least one antibody in the biological sample; and (b) comparing an immunosignature of the subject to one or more immunosignature standards, wherein the subject is classified as being likely to respond to IT treatment based on the comparison. The one or more immunosignature standards can comprise at least one immunosignature of a subject or subjects known to respond to IT treatment. In some cases, the comparison is predictive of the clinical outcome of the treatment in the subject. The method can further comprise quantifying the off-target binding of the at least one antibody of the sample to one or more peptides of the plurality to form the immunosignature. The plurality of peptides can be a random peptide array. The biological sample can be a blood, tissue, or other bodily sample. The blood sample can be a peripheral blood sample. The immunotherapeutic can be selected from the group consisting of a CTLA-4 inhibitor, a PD-L1 inhibitor, and a PD-1 inhibitor. The cancer can be selected from the group consisting of renal cell carcinoma, lung cancer, and melanoma. The cancer can be a recurrent cancer. The subject can be a mammal. The mammal can be a human. The mammal can be a canine.

In some cases, the method further comprises creating a record indicating the subject is likely to respond to the checkpoint inhibitor treatment based on the immunosignature. The record can be created on a computer-readable medium.

These and other features, aspects, and advantages described herein will become better understood by persons of ordinary skill in the art upon consideration of the following drawings, detailed description, and appended claims.

### BRIEF DESCRIPTION OF THE DRAWINGS

The following drawings form part of the present specification and are included to further demonstrate certain illustrative aspects of the methods provided herein, which may be better understood by reference to one or more of these drawings in combination with the detailed description of specific embodiments presented herein. Persons of ordinary skill in the art will readily recognize and appreciate that the drawings are non-limiting and presented for exemplary purposes.

FIG. 1 is a schematic illustrating the use of immunosignatures (IMS) or frameshift signatures to (1) predict a subject's response to immunotherapeutics (IT) therapy before and during the treatment, (2) predict and monitor serious side-effects of ITs, and (3) provide a prognosis regardless of treatment. Information for these analysis can guide whether to use an IT of not and which IT to use.

FIG. 2 illustrates two types of arrays that can be used for these distinctions: (left) Immunosignature arrays are created with about 10,000 to 1,000,000 peptides that are chosen from random sequence space. The peptides are 8-30 amino acids long are spaced to create avidity binding of antibodies for lower affinity epitopes in the peptides. (right) Frameshift arrays are created with about 100-400K peptides that are chosen from the 220,000 possible FS peptides resulting from indels in microsatellites (inserted in the DNA or only the RNA) or from mis-splicing of exons in forming the RNA. The peptides are 8-30 amino acids long and are spaced further than in IMS to enhance high affinity, cognate binding of antibodies.

FIGS. 3A-3B demonstrate that IMS can distinguish between responders to an anti-PDL1 and anti-CTLA4 combination treatment in an osteosarcoma lung metastasis mouse model. (A) After i.v. injection of K7M2 tumor cells, the mice were treated with 3 dosages of anti-PD-L1 plus anti-CTLA4 and 2 more dosages of anti-PD-L1 treatment. Each treatment was three days apart. (B) The IMS were analyzed (1) before the tumor injection and the treatment, (2) right after the treatment, and (3) at the end of the experiment when the non-respond mice died from lung metastasis. At each time point, the IMS significantly distinguished responder mice (green bar) and non-responder mice (red bar).

FIG. 4 is a graph demonstrating the percentage tumor-free mice per treatment group over time. Non-treatment group: no treatment before palpable tumor (28 mice). Early treatment group: 16 mice; late treatment group: 15 mice. Non-treatment vs Early treatment: p-value=0.0322; Non-treatment vs Late treatment: p-value=0.0003.

FIG. 5 is a heat-map demonstrating hierarchical clustering of 120,000 immunosignatures (IMS) for responders (green bar) vs non-responders (red bar) in the early treatment group.

FIG. 6 is a graph presenting Principle Component Analysis (PCA) data for responder and non-responder groups. Green dots: Responder; Red dots: Non-responder.

FIG. 7 demonstrates hierarchical clustering of peptides distinguishing early (red bar) and late tumor (green bar) events in the non-treatment group. Only one late tumor event was misclassified. This indicates that an IMS can distinguish the course of a tumor (prognosis) regardless of treatment.

FIG. 8 demonstrates hierarchical clustering of peptide distinguishing Responders (green bar) vs Non-responders (red bar) in the early treatment group using 33 significant FS peptides from an 800 FS peptides array.

FIG. 9 that the IMS can be used to distinguish human cancer patients that responded (n=10, green bar) or did not (n=20, red bar) to IT treatment. Leave one out validation shows about 79% accuracy to predict the responder to the IT treatment.

FIGS. 10A-10D demonstrate that FS peptide signatures can be used to distinguish the responses of human cancer patients to IT treatment. Serum samples from responders (n=10, green bar), non-responders (n=20, red bar) and 20 healthy subjects (n=20, blue bar) were applied to a FS peptide array. Each panel uses a different classification method. (A). Selection of 200 most significantly higher IgG reactive FS peptides in responders based on t-test by comparing to non-responders could cluster responders and non-responders. Leave one out validation with SVM shows 96.6% accuracy to predict the responder to the IT treatment. (B). Using the total relative fluorescence units (RFU) of the selected 200 significant FS peptides to predict the responder. The total RFU greater than 1,000, is the responder (green bar); which the total RFU less than 400, is the non-responder (red bar) or healthy subject (blue bar). The accuracy of leave-one-out validation of this method is 96%. (C). Cluster the responder (green bar) and non-responder (red bar) by highly positive reactive FS peptide. Positive cut-off value was calculated by average of the non-responders plus 6 fold of the standard deviation of the non-responders. Selection of the top 500 positive peptides in responders can cluster 100% accuracy of the responders and non-responders. (D). Using the positive peptide number to predict the responder patients to the IT treatment. Total positive peptide number was counted of each sample from the top 500 positive rate FS peptides of the responders. Positive cut-off value was calculated by average of the non-responders (NR) plus 6 fold of the standard deviation of the non-responders. 20 rounds leave-one-NR-out validation decided the cut-off positive peptide number for responder is 79 (average plus 95% CI of the NR positive peptide number). The average accuracy to predict the responder is 97.3%. Error bar is 95% CI.

FIG. 11 is a heat-map demonstrating that FS peptide signatures can distinguish the patients likely to experience serious side-effects (blue) under IT treatment from the “no side-effect” patients (red). Significant FS peptides were selected by comparing side-effect patients and no side-effect patients. Heat-map shows that the selected FS peptides cluster the side effect patients with 100% accuracy.

FIGS. 12A-12B demonstrates that FS peptide arrays can distinguish patients likely to have a serious side-effect (blue) under IT treatment from the “no side effect” patients (red). Significant FS peptides were selected by comparing side-effect patients and no-side effect patient patients. Both heat-map (A) and Principle Component Analysis (B) demonstrated that the selected FS peptides cluster the side effect patients with 100% accuracy.

### DETAILED DESCRIPTION

All publications, patents, and patent applications mentioned in this specification are herein incorporated by reference to the same extent as if each individual publication, patent, or patent application was specifically and individually indicated to be incorporated by reference.

This disclosure, by way of certain illustrative and non-limiting examples, provides methods and compositions (e.g., peptide arrays and kits) for predicting the response of a subject (such as a human or animal patient) to checkpoint inhibitor therapies. The methods and compositions provided herein are based at least in part on the appreciation that tumor mutational burden (TMB) is not reliably correlated with responsiveness to checkpoint inhibitor therapy. It was determined that the breadth and content provided by immunosignatures can be used to identify which patients are likely to respond favorably to an IT therapy and to identify subjects that are likely to be non-responsive or have adverse reactions to this form of cancer treatment.

Without being bound by any particular theory or mechanism, it is believed that the immunosignature (IMS) of a tumor better reflects the antigens being presented by the tumor to the cancer patient's immune system and that the IMS is a better correlate than mutational load for assessing responsiveness to treatment. Accordingly, IMS provides a simple and reliable method for determining with high confidence which patients are likely to respond (e.g., to respond favorably from a clinical perspective) to IT therapy. Advantageously, immunosignaturing is a more direct method of identifying subjects likely to respond to IT treatment than assessing receptor density on tumor cells or sequencing tumors to determine mutational load.

A second type of peptide array, a frameshift (FS) peptide array, is also effective in making these clinical distinctions. Unlike the IMS, FS arrays are based on the prediction of specific peptides that will be immunoreactive in cancer. It is known that tumor cells are much more prone to insert or delete nucleotides in microsatellites (thus creating indels)—both when replicating DNA and transcribing RNA—than non-cancer cells. Tumor cells are also more likely to mis-splice exons. Indels and mis-splice errors create FS peptides which are highly immunogenic. If these peptides are important in the immune response of the patient to the tumor, they are likely to be important in determining the response to IT therapy, prognosis, and the likelihood that a particular subject will experience serious side effects to IT therapy. Therefore, antibodies to these peptides are more likely to correlate with IT responses than TMB.

### Methods

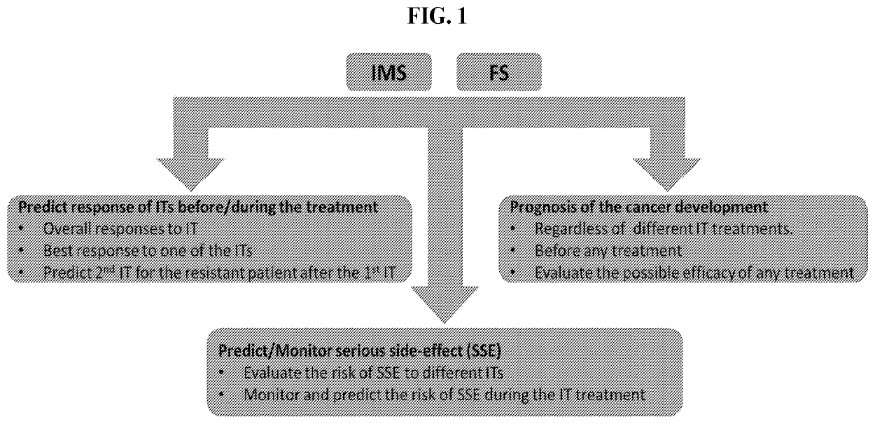

Accordingly, in a first aspect, provided herein is a method of classifying how a subject having cancer is likely to respond to IT therapy and, as described herein, can include, for example, obtaining the subject's immunosignature (IMS) or Frameshift (FS) signature (or multiple immunosignatures or FS signatures) considered independently or in combination) using one or more biological samples obtained from the subject to determine whether the sample contains one or more indicators of favorable or unfavorable responses (e.g., unfavorable side effects) to IT therapy. As illustrated in FIG. 1, this disclosure also provides methods for classifying a subject as having a good prognosis or a poor prognosis for survival, and methods monitoring the effectiveness of a course of IT treatment for a subject having cancer.

The correlation between an IMS and responsiveness to IT therapy can be established by obtaining IMS profiles for subjects having a known favorable response to IT treatment and for subjects that were unresponsive or had an unfavorable response to treatment using sera (or other bodily samples) collected before each subject received treatment. In some cases, an IMS control includes non-disease sera contacted with an identical array under the same experimental conditions. The breadth of the IMS can be quantified in multiple ways including, for example the number of motifs, the percentage of signature represented, and/or total immune reactivity. Once the quantitative correlate is been established, cancer patients can be classified according to a method provided herein by quantifying a subject's signature for responsiveness to IT treatment, prognosis, or likelihood of experiencing serious side-effects of IT treatment.

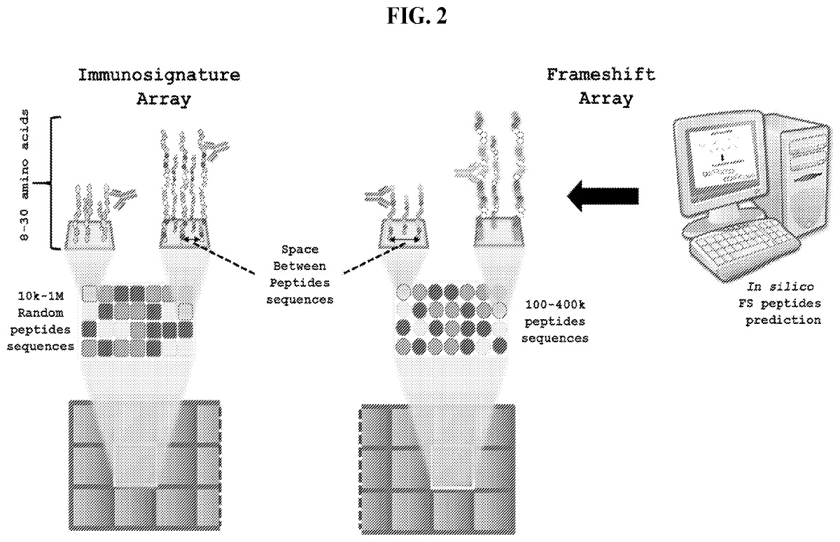

Frameshift (FS) signatures provide another mechanism for classifying patients according to their responsiveness to IT treatment, prognosis, or likelihood of experiencing serious side-effects of IT treatment. In contrast to the IMS arrays, FS arrays comprise peptides selected from a finite set of approximately 220,000 possible peptides. Frameshift peptides are derived from indels in microsatellites and mis-splicing of exons. These FS peptide collections for humans and dogs have been disclosed in U.S. Patent Publication No. 2014/0087963 (filed Aug. 29, 2013), which is incorporated herein by reference as if set forth in its entirety. FS arrays can be constructed in the same manner as the IMS arrays except that, as illustrated in FIG. 2, the space between each peptide is increased to enhance cognate binding rather than mimotope binding as in IMS arrays. The sequence length of the FS peptides chosen for the array is generally 8-60 amino acids (AA) long. Peptides longer than 15 AA are represented by 2 or more peptides on the array. Accordingly, approximately 400,000 15-AA peptides on the array can represent most of the 220,000 possible FS peptides.

In certain embodiments, the methods provided herein comprise immunosignaturing (i.e., the process of detecting immunosignatures). For additional description of immunosignaturing, see U.S. Patent Publication No. 2014/0087963 (filed Aug. 29, 2013), which is incorporated herein by reference as if set forth in its entirety. Immunosignaturing displays a circulating antibody repertoire upon an addressable, machine-readable peptide microarray (e.g., a random peptide microarray). The dynamics of circulating antibodies includes both the presence and the absence of an antibody or a plurality of antibodies from the system of a subject. The random sequences allow an unbiased display of all types of antibody binding. The peptides on the microarray serve as mimetics of the actual epitopes and capitalize on the cross-reactivity of antibodies. Even if the actual epitope is not present, another peptide that the same antibody can bind will be present. In addition, the arrays are inexpensive and can be adapted to high-throughput sample processing.

Immunosignatures (IMS) are a merger of microarray and phage technologies that display the complexity of the humoral immune response and convert it into a machine-readable, quantitative format. Immunosignatures are produced by profiling the antibody repertoire of an individual on a chip arrayed with non-natural sequence peptides. It is attractive in that it is a simple but comprehensive measure of the complexity of the humoral response. Immunosignatures detect even tiny perturbations in health status early and accurately. Comprehensive measurements of antibody repertoires provide the means for rapid, inexpensive, and early diagnosis of any diseased state; ultimately, the continuous monitoring of immunosignatures may provide the means to detect dangerous disease states pre-symptomatically.

Generally, immunosignaturing comprises contacting a biological sample (e.g., blood or other bodily sample) with a large number of peptides, nucleic acids, or other biomolecules, where each biomolecule is associated with a feature on a surface. Antibodies in the sample bind differentially to the query molecules at each feature, thus forming a pattern of binding that provides a detailed insight into the molecular recognition profile of the antibodies in the blood. See, e.g., Stafford and Johnston, Exp. Rev. Aloi. Diagn. 11:5-8 (2011). Immunosignatures detect and partition an antibody response into a coherent set of signals that can be mathematically interpreted. The concept is that any change in health is likely to be represented by a change in this molecular recognition profile. Such profiles can be used in various analytical methods to further characterize the sample. See for example, U.S. Patent Publication No. 2013/0079242, which is incorporated herein as if set forth in its entirety. Thus, immunosignatures are distinct from and an alternative to traditional, individual protein or genetic biomarkers for the diagnosis of various conditions.

In certain embodiments, the method presented herein comprises contacting a biological sample (e.g., a complex biological sample) of a subject to a peptide array, wherein the peptide array comprises a plurality of peptides capable of off-target binding of at least one antibody in the biological sample; measuring the off-target binding of the antibody to a group of different peptides in the peptide array to form an immunosignature (IMS); and associating the IMS with responsiveness to a particular treatment (e.g., administration of a particular IT) or with a state of health. As used herein, the terms “off-target binding” and “off-target antibody binding” are used interchangeably and refer to unique binding interactions between high affinity monoclonal antibodies and random peptide sequences. The varying strengths of these unique binding reactions can be measured and compared. See U.S. Patent Publication No. 2014/0087963, which is incorporated herein as if set forth in its entirety. See also Halperin et al., Exploring Antibody Recognition of Sequence Space through Random-Sequence Peptide Microarrays. Molecular & Cellular Proteomics 10 (2011). Curiously, many of these off-target mimotope interactions had higher binding than the cognate epitope. Although the corresponding solution-phase binding of these interactions is low, the way the immunosignature microarray is constructed enhances these interactions.

A FS signature is established by using a biological sample (e.g., blood, sera, plasma) that may contain antibodies having affinity to peptides on the FS array. As described herein, antibodies are employed as biomarkers of disease, thus taking advantage of the immune system's expansive antibody repertoire to identify a statistically significant pattern of peptides, each with specific binding values having predictive, prognostic, and diagnostic potential. In some cases, the biological sample is diluted. The sample is incubated long enough to allow cognate binding to approach equilibrium—usually overnight. The array is washed and then incubated with secondary antibody to quantify the amount of antibody bound to each peptide on the array. For each peptide a quantitative amount of fluorescence is determined. These quantitative data can be analyzed in many different analytical and statistical approaches. In general, a patient's FS signature for IT response, prognosis, or side-effects is determined by comparing two or more groups of interest. For example, a comparison may be made between patients who responded well to IT therapy and those that did not. Such comparisons are used to establish the classifier of interest. In some cases, because the FS arrays are directly measuring the immune response to tumor antigens, the difference in groups may be determined directly by quantifying total binding to the FS peptides.

In certain embodiments, the peptide array is a plurality of short linear peptides immobilized on a solid surface (e.g., a polystyrene or other solid substrate). As used herein, the terms “peptide” and “polypeptide” refer to a polymer in which the monomers are alpha amino acids joined together through amide bonds. Peptides are two or often more amino acid monomers long. Standard abbreviations for amino acids are used herein (see Stryer, 1988, Biochemistry, Third Ed., incorporated herein by reference). In certain embodiments, random-sequence peptide arrays are used. As used herein, the term “random peptide” refers to an oligomer composed of two or more amino acid monomers chosen from random sequence space but specifically synthesized. As used herein, the term “random peptide array” includes a set of such peptides as well as a set of fusion proteins containing such random peptides.

In some cases, the peptide array comprises a plurality of human frameshift peptides. In certain embodiments, the frameshift peptides comprise tumor-specific frameshift antigens (molecular targets). In such cases, the methods are useful for determining a subject's responsiveness for IT treatment of a tumor (including early stage tumor formation) associated with a frameshift mutation, which generally arise from mutations in the DNA of coding microsatellite regions or during transcription through microsatellites or mis-splicing. For example, a tumor-associated gene may harbor one or more coding microsatellite regions that, when a mutation occurs that leads to a frameshift with respect to the translational reading frame of the downstream nucleic acid sequence, may give rise to frameshift peptides. Alternatively, when this gene is transcribed a base is inserted or deleted forming a variant RNA that encodes a FS peptide. In addition, FS peptides can be formed through mis-splicing of exons which alter the reading frame.

In some cases, the methods provided herein involve multiplexed arrays in which a plurality of peptides or polypeptides (i.e., proteins) attached to a solid support are contacted to a biological sample (e.g., blood or other bodily tissue obtained from a subject).

Any suitable peptide array can be used on which the peptides are immobilized to a substrate. In some embodiments, the array comprises between 500-1,000,000 peptides; between 500-500,000 peptides; between 500-250,000 peptides; between 500-100,000 peptides; between 500-50,000 peptides; or between 500-10,000 peptides. In some embodiments, the peptides are 8-35, 12-35, 15-25, 10-30, or 9-25 amino acids in length. In some embodiments, the amino acid sequences of the peptides are randomly selected. In some embodiments, the pattern of amino acids present in the microarray is pre-defined (at least in part), and the array is not a random peptide array. In some cases, random-sequence peptide arrays used according to the methods provided herein comprise about 10,000 or more random sequence peptides.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethacrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivatization does not eliminate detection of binding between the peptides and antibodies in the sera.

As used herein, the term “sample” means non-biological samples and biological samples. Non-biological samples include those prepared in vitro comprising varying concentrations of a target molecule of interest in solution. Biological samples include, without limitation, blood, lymph, urine, saliva, sputum, other bodily secretions, cells, and tissue specimens and dilutions of them. Any suitable biological sample can be used. For example, a biological sample can be a specimen obtained from a subject (e.g., a mammal such as a human, canine, mouse, rat, pig, guinea pig, cow, monkey, or ape) or can be derived from such a subject. A subject can provide a plurality of biological sample, including a solid biological sample, from for example, a biopsy or a tissue. In some cases, a sample can be a tissue section or cells that are placed in or adapted to tissue culture. A biological sample also can be a biological fluid such as urine, blood, plasma, serum, saliva, tears, or mucus, or such a sample absorbed onto a paper or polymer substrate. A biological sample can be further fractionated, if desired, to a fraction containing particular cell types. In some embodiments, a sample can be a combination of samples from a subject (e.g., a combination of a tissue and fluid sample). In some cases, sera are obtained from the individual using techniques known in the art.

The methods provided herein are sensitive and involve small quantities of biological samples from a subject. In some embodiments, biological samples from a subject are too concentrated and require a dilution prior to being contacted with an array of the invention. A plurality of dilutions can be applied to a biological sample prior to contacting the sample with an array of the invention. A dilution can be a serial dilution, which can result in a geometric progression of the concentration in a logarithmic fashion. For example, a ten-fold serial dilution can be 1 M, 0.01 M, 0.001 M, and a geometric progression thereof A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

The binding of a molecule to an array in accordance with certain embodiments of the methodology disclosed herein creates a pattern of binding that can be associated with a condition. The affinity of binding of a molecule to a peptide in the array can be mathematically associated with a condition. The off-target binding pattern of an antibody to a plurality of different peptides of the invention can be mathematically associated with a condition. The avidity of binding of a molecule to a plurality of different peptides can be mathematically associated with a condition. The off-target binding and avidity can comprise the interaction of a molecule in a biological sample with multiple, non-identical peptides in a peptide array. An avidity of binding of a molecule with multiple, non-identical peptides in a peptide array can determine an association constant of the molecule to the peptide array. In some embodiments, the concentration of an antibody in a sample contributes to an avidity of binding to a peptide array, for example, by trapping a critical number or antibodies in the array and allowing for rapid rebinding of an antibody to an array.

The peptide array can be contacted with the biological sample (e.g., sera) under any suitable conditions to promote binding of antibodies in the sample to peptides immobilized on the array. Thus, the methods presented herein are not limited by any specific type of binding conditions employed. Such conditions will vary depending on the array being used, the type of substrate, the density of the peptides arrayed on the substrate, desired stringency of the binding interaction, and nature of the competing materials in the binding solution. In a certain embodiments, the conditions comprise a step to remove unbound antibodies from the addressable array.

Similarly, any suitable detection technique can be used in the methods provided herein to detect binding of antibodies in the biological sample to peptides on the array to generate a subject's immunosignature or FS signature. In one embodiment, any type of detectable label can be used to label peptides on the array, including but not limited to radioisotope labels, fluorescent labels, luminescent labels, and electrochemical labels (i.e., ligand labels with different electrode mid-point potential, where detection comprises detecting electric potential of the label). Alternatively, bound antibodies can be detected, for example, using a detectably labeled secondary antibody.

The composition of molecules in an array can determine an avidity of binding of a molecule to an array. A plurality of different molecules can be present in an array used to predict a subject's responsiveness to a particular treatment. Non-limiting examples of biomolecules include amino acids, peptides, peptide-mimetics, proteins, recombinant proteins antibodies (monoclonal or polyclonal), antibody fragments, antigens, epitopes, carbohydrates, lipids, fatty acids, enzymes, natural products, nucleic acids (including DNA, RNA, nucleosides, nucleotides, structure analogs or combinations thereof), nutrients, receptors, and vitamins. In some embodiments, a molecule in an array is a mimotope, a molecule that mimics the structure of an epitope and is able to bind an epitope-elicited antibody. In some embodiments, a molecule in the array is a paratope or a paratope mimetic, comprising a site in the variable region of an antibody (or T-cell receptor) that binds to an epitope of an antigen. In some embodiments, an array employed in accordance with the methodologies presented herein is a peptide array comprising random peptide sequences or known frameshift peptides.

In certain embodiments, the subject has been diagnosed with cancer or other cell proliferative disorder. As used herein, the term “cancer” refers to the broad class of disorders characterized by hyperproliferative cell growth, either in vitro (e.g., transformed cells) or in vivo. Cancers appropriate for treatment with checkpoint inhibitor therapy include without limitation a variety of neoplasms, including benign or malignant tumors, a variety of hyperplasia, and the like. Non-limiting examples of cancers that can be diagnosed, monitored, prevented, and/or treated with an array and a method of the invention can include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor. The cancer could be diagnosed by an IMS or FS signature, even at a very early stage.

As used herein the term “immunotherapeutic” or “IT” refers to a compound that is used to, in this case, treat cancer by inducing, enhancing or suppressing the immune response. Immunotherapeutics encompass immune checkpoint inhibitors, antibody-drug conjugates (ADCs), monoclonal antibodies, T-cell therapy, small molecules, and bispecific antibodies (bsAbs). Antibody-drug conjugates include monoclonal antibodies linked to biologically active drugs to combine the targeting ability of antibodies as well as the cytotoxic ability of the drug. T-cell therapy involves reprogramming a patient's own immune T-cells to attack tumors. One type of well-known T-cell therapy comprises adoptive transfer of chimeric antigen receptor (CAR) T-cells. As used herein, the term “chimeric antigen receptor” refers to a fusion protein of the membrane or intracellular signaling region of T-cell activating proteins (e.g., CD3-zeta chain, CD28, 41BBL, OX40, ICOS, high-affinity receptor for IgE (FcεRI) and other T-cell activating proteins) and the antigen-binding site (i.e., single-chain Fv fragment) of a cancer antigen-specific antibody. Bispecific antibodies are recombinant proteins that can bind to two different types of antigen at the same time. For example, a bsAb can be engineered to bind a cytotoxic cell and a target tumor cell. That way, the bsAb brings the cytotoxic cell and the target tumor cell into close proximity and facilitates tumor treatment.

In certain embodiments, the immunotherapeutic is selected from Tremelimumab (CTLA-4 blocking antibody), OX40 agonists (e.g., agonist antibodies), antibodies to B7 ligands (e.g., anti-B7-H1, anti-B7-H3, anti-B7-H3, anti-B7-H4), durvalumab (MEDI4736, anti-PD-L1 antibody), MK-3475 (PD-1 blocker), Nivolumab (anti-PD-1 antibody), Pembrolizumab (anti-PD-1 antibody), Pidilizumab/CT-011, BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody), and Yervoy/ipilimumab (anti-CTLA-4 checkpoint inhibitor). Many new inhibitor targets are being investigated. In some cases, IT treatment comprises a combination therapy in which two or more immunotherapeutics are administered.

As used herein the terms “checkpoint inhibitor” and “checkpoint pathway inhibitor” are used interchangeably and refer to negative regulatory molecules, usually antibodies, that block or inhibit anti-T-cell anti-tumor function to enhance tumor killing. Checkpoint inhibitors include, without limitation, CTLA-4, PD-L1, PD-L2, PD-1, B7-H3, B7-H4, BTLA, HVEM, TIM3, GAL9, LAG3, VISTA, KIR, 2B4, CD160, CGEN-15049, CHK1, CHK2, A2aR, and a B-7 family ligand such as B7-1, B7-2, B7-DC, B7-H1, B7-H2, B7-H3, B7-H4, B7-H5, B7-H6 and B7-H7 (or any combination thereof), or a combination thereof (e.g., a combination of CTLA-4 and PD-L1 or PD-L2).

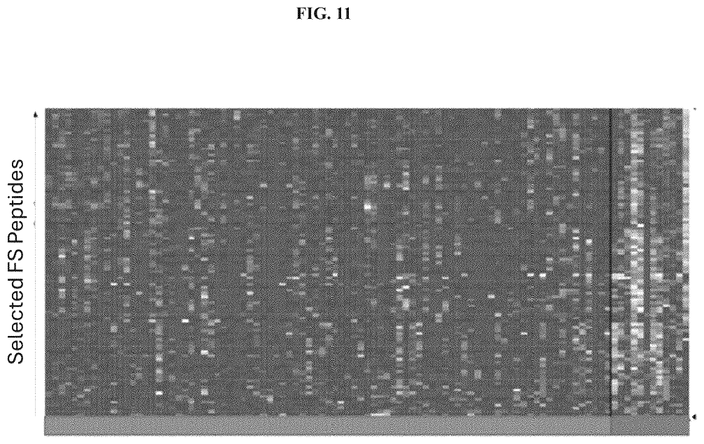

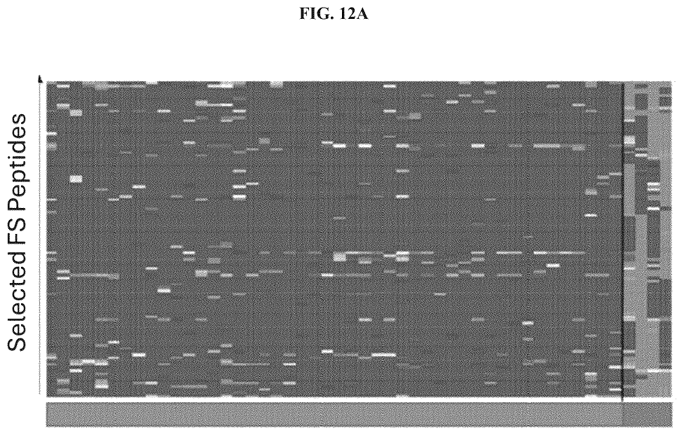

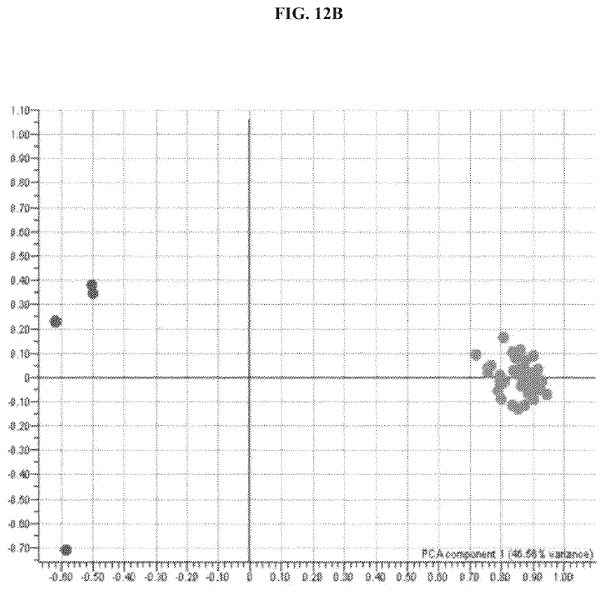

As used herein, the term “side effect” or “side effects” refers to the unacceptable or undesirable adverse symptoms resulting from or associated with the administration of a particular treatment such as an IT therapy. Side effects specifically to immunotherapeutics are termed “immune related adverse events” (irAE). While side effects vary by the type of therapy, common side effects of IT therapies include, without limitation fatigue, infusion related reactions, dermatological toxicity, diarrhea/colitis, hepatotoxicity, pneumonitis, hyper-and hypo-thyroidism. For review, see e.g., uptodate.com/contents/patient-selection-criteria-and-toxicities-associated-with-checkpoint-inhibitor-immunotherapy on the World Wide Web. Immune-related adverse events are generally graded from 1-4. Grades 3 and 4 are considered serious and can require immunosuppression treatment. Patients with irAE are just as likely to have a positive response to treatment. Occurrence of Grade 3 or 4 event can prohibit the patient from further IT therapy. Therefore, knowing ahead of time which patients are more likely to have an event would allow closer monitoring to pre-empt a Grade 3 or 4 event. The irAE patients in FIG. 11 experienced grade 2 or higher events (e.g., hypothyroidism, diarrhea, elevated ALT/AST (hepatotoxicity), colitis, diabetes, rash, fatigue). It is of note that, even though they suffered from a variety of events, they had a common predictive signature. Patients associated with samples reported in FIG. 11 suffered from grade 2 or higher events such as hypothyroidism, diarrhea, elevated ALT/AST (hepatotoxicity), colitis, diabetes, rash, and fatigue.

Any appropriate criteria can be used to confirm a subject's responsiveness to treatment with an IT. For example, in certain embodiments, responsiveness to treatment by an IT is measured by at least one criterion selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

The methods described herein can be carried out using a computer programmed to receive data (e.g., data from a random or FS peptide array indicating whether a subject has a signature associated with responsiveness to IT therapy). The computer can output for display information related to a subject's biomarkers, and the likelihood of the duration of time that the subject will be responsive to an IT therapy, suffer a side-effect, or the prognosis of survival.

After information regarding a subject's biomarkers is reported, a professional can take one or more actions that can affect patient care (e.g., administer a new treatment or modify an existing treatment). For example, a medical professional can record the information in a subject's medical record and/or in an electronic database. In some cases, a medical professional can record that the subject is likely or not likely to respond to an IT therapy, or otherwise transform the patient's medical record, to reflect the patient's medical condition. In some cases, a medical professional can review and evaluate a patient's medical record, and can assess multiple treatment strategies for clinical intervention of a patient's condition. The signature may indicate watchfulness or pre-treatment for a side-effect or recommendation for a different treatment.

A professional (e.g., medical professional) can communicate information regarding biomarker analysis to a subject or a subject's family. In some cases, a professional can provide a subject and/or a subject's family with information regarding an IT therapy, including treatment options and potential side effects. In some cases, a professional can provide a copy of a subject's medical records to communicate information regarding biomarker analysis and/or disease states to a specialist.

A professional (e.g., research professional) can apply information regarding a subject's biomarkers to advance research into IT therapy. For example, a researcher can compile data on the presence of a particular signature with information regarding the efficacy of an IT therapy, or side effects associated with an IT therapy. In some cases, a research professional can obtain a subject's biomarker information to evaluate the subject's enrollment, or continued participation in a research study or clinical trial. In some cases, a research professional can communicate a subject's biomarker information to a medical professional, or can refer a subject to a medical professional for clinical assessment and/or treatment.

Any appropriate method can be used to communicate information to another person (e.g., a professional), and information can be communicated directly or indirectly. For example, a laboratory technician can input biomarker information into a computer-based record. In some cases, information can be communicated by making a physical alteration to medical or research records. For example, a medical professional can make a permanent notation or flag a medical record for communicating information to other medical professionals reviewing the record. Any type of communication can be used (e.g., mail, e-mail, telephone, and face-to-face interactions). Information also can be communicated to a professional by making that information electronically available to the professional. For example, information can be placed on a computer database such that a medical professional can access the information. In addition, information can be communicated to a hospital, clinic, or research facility serving as an agent for the professional.

### Articles of Manufacture

This disclosure also provides articles of manufacture that can include, for example, materials and reagents that can be used to determine whether a subject has a biomarker for predicting response to an IT treatment. An article of manufacture can include, for example, peptides, nucleic acids, or polypeptides immobilized on a substrate (e.g., in discrete regions (“features”) with different populations of isolated peptides, nucleic acids, or polypeptides immobilized in each discrete region) such as in a nucleic acid array. The article of manufacture can also include instructions for use in practicing a method for predicting the likelihood of a subject responding to an IT treatment as provided herein.

The article of manufacture may further comprise one or more nucleic acid arrays or peptide arrays for performing the analysis. In some cases, the nucleic acid arrays and peptide arrays are attached to a solid substrate, e.g., a porous or non-porous material that is insoluble. The nucleic acids or peptides of each array can be immobilized on the substrate covalently or non-covalently.

Also provided are kits containing any of the nucleic acid arrays described herein. The kits can optionally contain instructions for detecting one or more signatures described herein. The kits can optionally include, e.g., a control biological sample or control labeled-amplicon set containing known amounts of one or more amplicons recognized by nucleic acid probes of the array.

In some cases, one or more reagents for processing a biological sample and/or using the arrays (e.g., reducing reagents, denaturing, deglycosylating reagents, dephosphorylating reagents, alkylating reagents and/or reagents for chemically or enzymatically cleaving a peptide or protein) are provided with the kit. A kit also can include a detection reagent for detecting the presence or absence of a particular signature. Alternatively, such reagents may be provided separately from the kit.

In some cases, the kits can include a software package for analyzing the results of, e.g., a peptide array analysis, immunosignaturing, or FS array analysis.

Instructions for the above-described articles of manufacture are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub packaging), etc. In other embodiments, the instructions are present as an electronic storage data file present on a suitable computer readable storage medium, e.g., CD-ROM, diskette, etc., including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The kits described herein also can optionally include instructions for treating a cancer patient based on the presence or absence of a signature as described herein.

“Determining,” “measuring,” “assessing,” “assaying” and like terms are used interchangeably and can include both quantitative and qualitative determinations. Assessing may be relative or absolute. “Assessing the presence of” includes determining the amount of something present, as well as determining whether it is present or absent.

Note that as used herein and in the appended claims, the singular forms “a,” “an,” and “the” include plural referents unless the context clearly dictates otherwise. Thus, for example, reference to “an array” refers to one or more such arrays, and reference to “the method” includes reference to equivalent steps and methods known to those skilled in the art, and so forth.

It is contemplated that any embodied method or composition described herein can be implemented with respect to any other method or composition described herein.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Although the embodiments are described in considerable detail with reference to certain methods and materials, one skilled in the art will appreciate that the disclosure herein can be practiced by other than the described embodiments, which have been presented for purposes of illustration and not of limitation. Therefore, the scope of the appended claims should not be limited to the description of the embodiments contained herein.

Certain embodiments of the invention are further described in the following example, which does not limit the scope of the invention described in the claims but rather is included to demonstrate such embodiments. It should be appreciated by those of skill in the art that the techniques disclosed in the examples which follow represent techniques developed by the inventors to function well in the practice of the methods provided herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1—Classifying Responders to Combination Immunotherapy Treatments

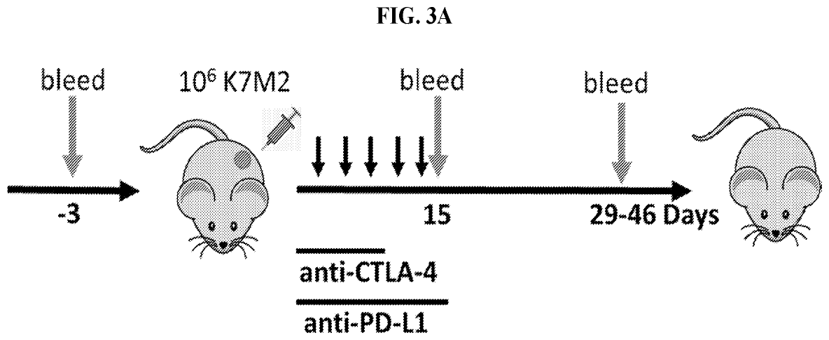

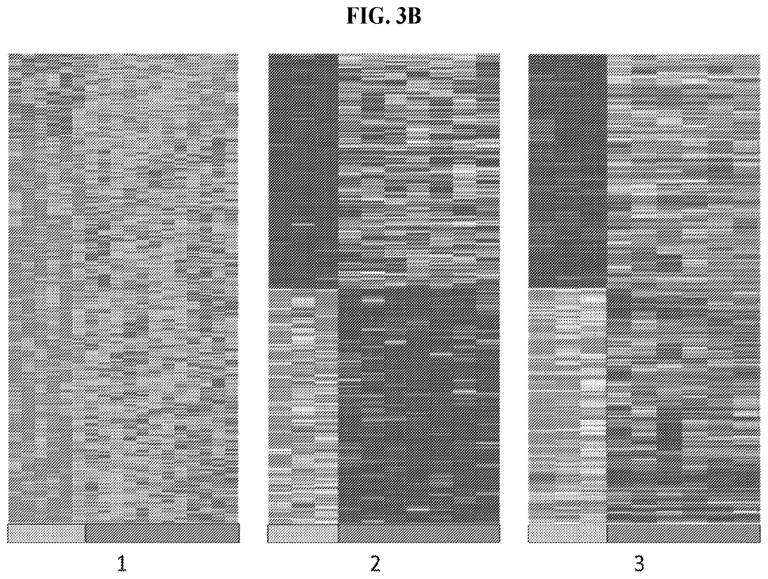

As shown in FIGS. 3A-3B, IMS can distinguish responders to anti-PDL1 and anti-CTLA4 combination treatment from non-responders in a mouse model of osteosarcoma lung metastasis. Following intravenous (i.v.) injection of cells of the K7M2 osteosarcoma cell line, the mice were treated with 3 dosages of anti-PDL1 plus anti-CTLA4, and 2 additional dosages of anti-PLD1 treatment. Treatments were spaced three days apart. IMSs were analyzed at the following time-points: (1) prior to the tumor injection and the treatment, (2) right after the treatment, and (3) at the end of the experiment when non-responder mice have died from lung metastasis. At each time point, IMS significantly distinguished the responder mice from the non-responder mice. Remarkably, IMS could predict the response even before the tumor was injected.

Further study of IT response prediction was performed using a mammary tumor mouse model and either an IMS or FS peptide array. The 4T-1 mammary tumor cell line was used. Four groups of mice were assayed. Group 1: No Treatment (28 mice). Group 2: Early treatment group (1^{st} treatment at 16 weeks) (16 mice). Group 3: Treat at first palpable tumor (1^{st} treatment at ˜33 weeks). Group 4: Late treatment group (1st treatment at 24-26 weeks) (15 mice). IT treatment was 100 μg anti-CTLA4 (UC10-4F10-11) plus 200 μg anti-PD-L1 (10F.9G2). Five doses were administered, with each dose administered every 3 days, and then two additional doses, with one every week. Palpable tumors were monitored following the treatment period.

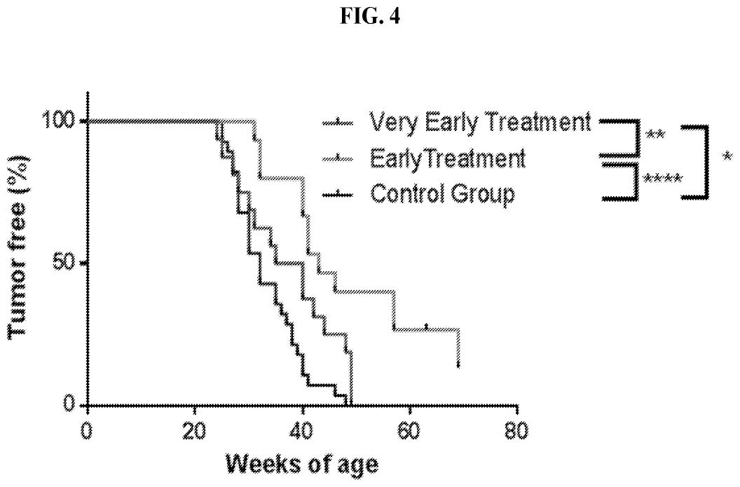

As shown in FIG. 4, palpable tumor initiation was significantly delayed by very early IT treatment and early treatment. Late IT treatment had no effect (data not shown). These results are presented in Table 1.

TABLE 1
Responder vs Non-responder in Early Treatment Group

| | Tumor Initiation | |
|---|---|---|
| Mouse | Age (weeks) | Type |
| ET5-6 | 23.7 | Non-responder |
| ET5-3 | 24.7 | Non-responder |
| ET3-2 | 27.14 | Non-responder |
| ET2-2 | 27.57 | Non-responder |
| ET3-3 | 30.14 | Non-responder |
| ET1-3t | 30.86 | Non-responder |
| ET3-6 | 33.57 | Non-responder |
| ET2-8 | 41.57 | Responder |
| ET2-10 | 47.57 | Responder |
| ET3-8 | 48.57 | Responder |
| ET1-5 | 48.86 | Responder |
| ET1-8 | 48.86 | Responder |

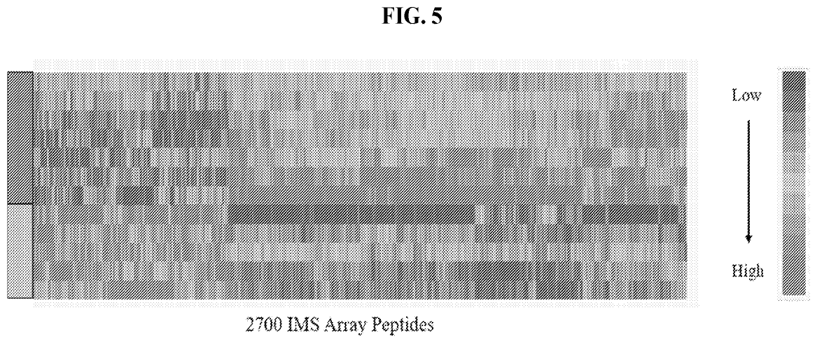

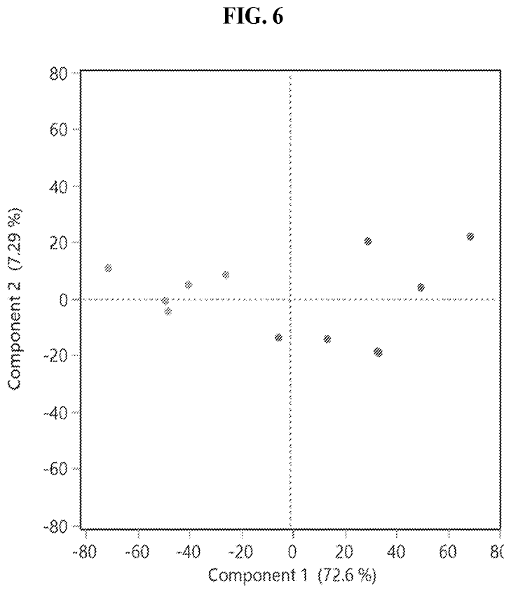

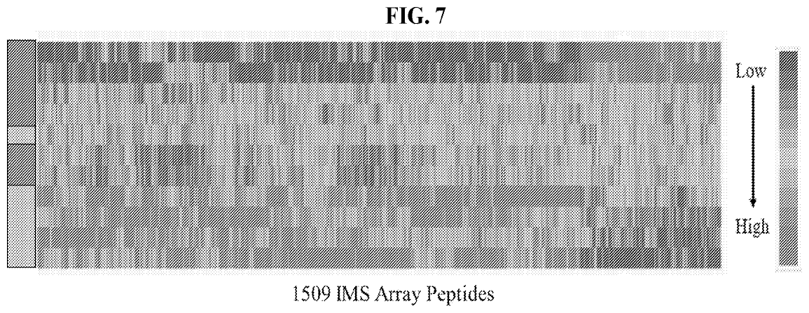

IMS from 120K peptide arrays were obtained for responders and non-responders in the early treatment group. As shown in FIGS. 5 and 6, 2700 peptides (selected by T-test) distinguished responders from non-responders in the early treatment group. It was further determined that the same 2700 peptides could distinguish early tumor events and late tumor events in the non-treatment group (see FIG. 7 and Table 2). This indicates that IMS arrays can distinguish the course of a tumor (prognosis) regardless of treatment.

TABLE 2
Early Tumor vs Late Tumor in Non-Treatment Group

| | Tumor Initiation | |
|---|---|---|
| Mouse | Age (weeks) | Type |
| NT3-7 | 24.57 | Early Tumor |
| NT1-2 | 25.14 | Early Tumor |
| NT4-6 | 26.14 | Early Tumor |
| TT5-4 | 27.14 | Early Tumor |
| TT5-6 | 27.14 | Early Tumor |
| TT3-5 | 27.57 | Early Tumor |
| NT3-3 | 39.57 | Late Tumor |
| TT4-3 | 39.57 | Late Tumor |
| NT1-Tat | 40.71 | Late Tumor |
| TT2-2 | 45.86 | Late Tumor |
| TT2-6 | 47.86 | Late Tumor |

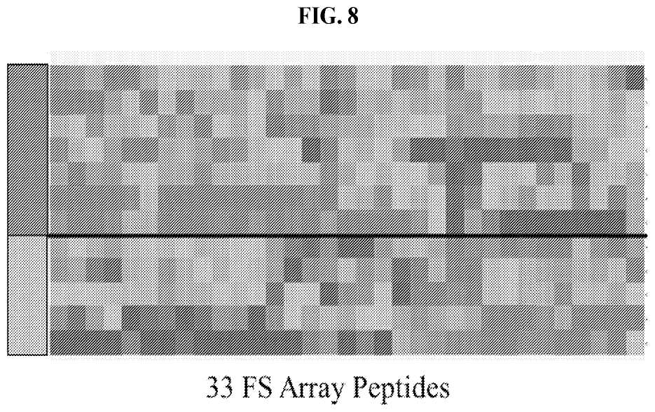

We also analyzed the same subjects (Responders versus Non-Responders) from the Early Treatment group on an 800-peptide, spotted FS array. As shown in FIG. 8, hierarchical clustering revealed 33 significant FS peptides from the 800-peptide FS array capable of distinguishing Responders (green bar) from Non-responders (red bar) in the early treatment group. These data demonstrate that, like IMS, FS peptide signatures are also able to distinguish Responders from Non-Responders.

### Example 2—Distinguishing Human Responders and Non-Responders to PD-1 Treatment Using IMS and FS Peptide Signatures

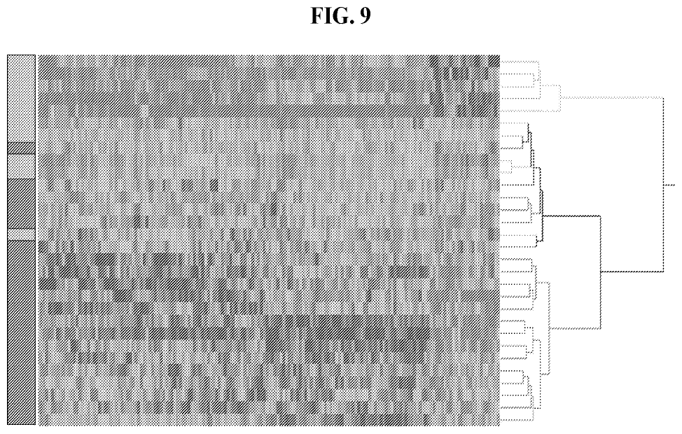

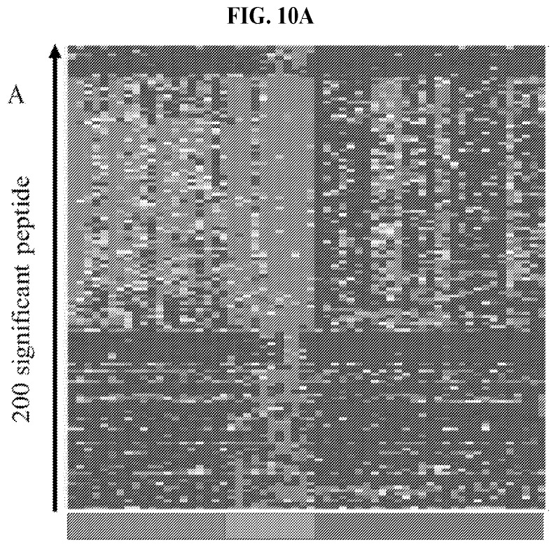

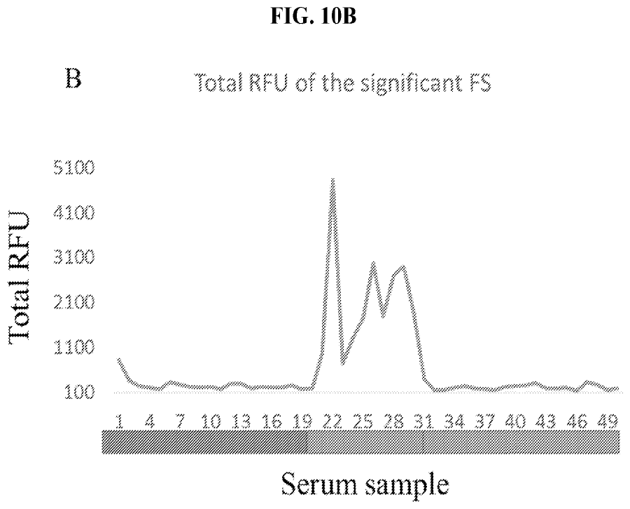

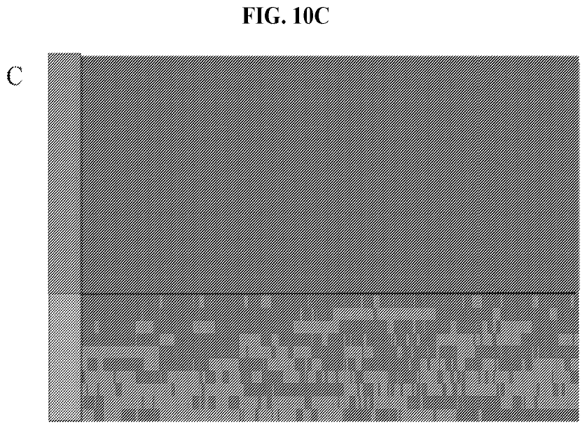

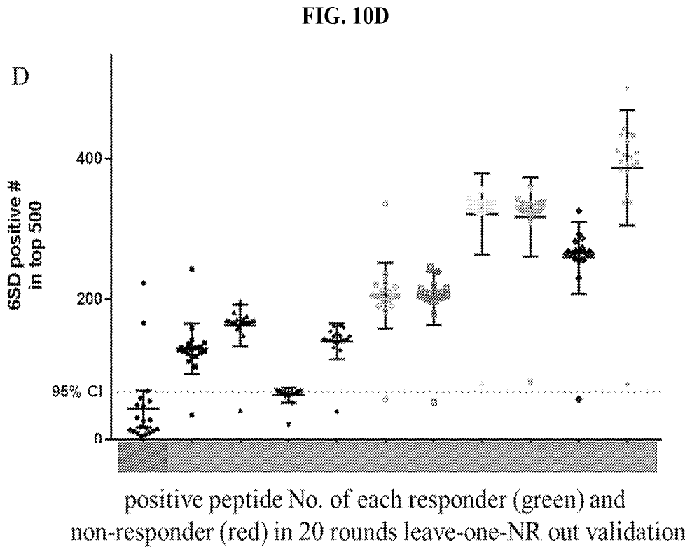

78 blood samples were obtained from MD Anderson. The samples were obtained from human patients having various cancers that were being treated with an immunotherapeutic agent that inhibits PD-1. The samples were obtained before treatment started. At the time of the IMS/FS assay, 30 patients had been monitored long enough to be designated as “Responder” or “Non-Responder” to PD-1 inhibitor treatment. All 78 samples were analyzed on the IMS arrays. As shown in FIG. 9, IMS can be used to distinguish human cancer patients that responded (n=10, green bar) or did not (n=20, red bar) to IT treatment. Leave one out validation revealed about 79% accuracy to predict responders to the IT treatment. The same set of 30 samples were also analyzed on FS arrays with 400K peptides. These arrays were even better at distinguishing the Responders from Non-Responders. Different types of analysis applied to the FS data yielded different accuracies (FIGS. 10A-10D). Based on this data, a physician could take a small sample of blood from a patient before treatment and determine with high accuracy whether the patient was likely to respond to that particular therapy. If the likelihood was low, a different therapy could be recommended. If the patient was predicted to be a responder but likely to have an immune-related adverse event (irAE) (FIG. 11), FS peptide signature analysis could be integrated into the treatment plan. irAE are generally graded from 1-4. Grades 3 and 4 are considered serious and can require immunosuppression treatment. Patients with irAE are just as likely to have a positive response to treatment. Occurrence of Grade 3 or 4 event can prohibit the patient from further checkpoint therapy. Therefore, knowing ahead of time which patients are more likely to have an event would allow closer monitoring to pre-empt a 3 or 4 event. The irAE patients in FIG. 11 experienced grade 2 or higher events (e.g., hypothyroidism, diarrhea, elevated ALT/AST (hepatotoxicity), colitis, diabetes, rash, fatigue). It is of note that, even though they suffered from a variety of events, they had a common predictive signature.

1. (canceled)

2. A method of measuring antibody affinity to a plurality of peptides, the method comprising:
(a) contacting a biological sample from a subject to a frameshift peptide array comprising a plurality of tumor-associated frameshift peptides that are 8-60 amino acids in length, wherein the frameshift peptides comprise frameshift peptides derived from indels arising during RNA transcription or from mis-splicing of exons;
(b) quantifying binding of antibodies in the contacted biological sample to one or more of the plurality of tumor-associated frameshift peptides; and
(c) generating a frameshift signature for the subject based on the quantified levels of binding to the plurality of the tumor-associated frameshift peptides.

3. The method of claim 2, wherein the frameshift signature is predictive of a clinical outcome of an immunotherapeutic treatment in the subject.

4. The method of claim 3, wherein the immunotherapeutic treatment is a cytotoxic T-lymphocyte-associated protein 4 (CTLA-4) inhibitor, a programmed death-ligand 1 (PD-L1) inhibitor, or a programmed cell death protein 1 (PD-1) inhibitor.

5. The method of claim 2, wherein the biological sample is a blood or tissue sample.

6. The method of claim 5, wherein the blood sample is a peripheral blood sample.

7. The method of claim 2, wherein the cancer is selected from the group consisting of breast cancer, renal cell carcinoma, lung cancer, and melanoma.

8. The method of claim 2, wherein the subject is a mammal.

9. The method of claim 8, wherein the mammal is a human.

10. The method of claim 7, wherein the mammal is a canine.

11. The method of claim 3, further comprising creating a record indicating the subject is likely to respond to the immunotherapeutic treatment based on the frameshift signature.

12. The method of claim 11, wherein said record is created on a computer readable medium.

13. A method of measuring a responder frameshift signature of a subject having a cancer, the method comprising the steps of:
(a) contacting a biological sample from the subject to a frameshift peptide array comprising a plurality of tumor-associated frameshift peptides that are 8-60 amino acids in length, wherein the frameshift peptides comprise frameshift peptides derived from indels arising during RNA transcription or from mis-splicing of exons;
(b) quantifying a level of binding of antibodies in the contacted biological sample to one or more of the tumor-associated frameshift peptides; and
(c) generating a responder frameshift signature based on the quantified level of binding of the antibodies, wherein the responder frameshift signature is indicative of whether the subject is a responder or non-responder to an immunotherapeutic treatment.

14. The method of claim 13, wherein the immunotherapeutic treatment is a cytotoxic T-lymphocyte-associated protein 4 (CTLA-4) inhibitor, a programmed death-ligand 1 (PD-L1) inhibitor, and a programmed cell death protein 1 (PD-1) inhibitor.

15. The method of claim 13, wherein the responder frameshift signature is predictive of the clinical outcome of the immunotherapeutic treatment in the subject.

16. The method of claim 13, wherein the biological sample is a blood or tissue sample.

17. The method of claim 16, wherein the blood sample is a peripheral blood sample.

18. The method of claim 13, wherein the subject is a mammal.

19. The method of claim 18, wherein the mammal is a human.

20. The method of claim 18, wherein the mammal is a canine.

21. The method of claim 13, further comprising creating a record on a computer readable medium, wherein the record indicates the subject is likely to respond to the immunotherapeutic treatment based on the frameshift signature.
